(19) United States
(12) Patent Application Publication
(10) Pub. No.: US 2023/0102138 A1
(43) Pub. Date: Mar. 30, 2023

(54) MEDICAL DEVICE FOR CUTTING A SUTURE DURING A MINIMALLY INVASIVE PROCEDURE

(71) Applicant: BOSTON SCIENTIFIC SCIMED, INC., MAPLE GROVE, MN (US)

(72) Inventors: Daniel Shuey, Pine City, MN (US); Christopher J. Koudela, New London, MN (US); Aaron Abbott, Columbia Heights, MN (US); Mitchell Nelson, Hudson, WI (US); Joel T. Eggert, Plymouth, MN (US); James K. Cawthra, JR., Ramsey, MN (US); Sandra L. Weeda, Scandia, MN (US)

(73) Assignee: BOSTON SCIENTIFIC SCIMED, INC., MAPLE GROVE, MN (US)

(21) Appl. No.: 17/953,748

(22) Filed: Sep. 27, 2022

Related U.S. Application Data
(60) Provisional application No. 63/249,683, filed on Sep. 29, 2021.

(51) Int. Cl. A61B 17/04

(52) U.S. Cl. CPC A61B 17/0467; A61B 17/0469

(57) ABSTRACT

A medical device for cutting a suture during a minimally invasive procedure may include an elongate shaft having a proximal end, a distal end, and a central longitudinal axis, a handle disposed at the proximal end of the elongate shaft, the handle including an actuation mechanism, and a cutting blade disposed proximate the distal end of the elongate shaft. The cutting blade is axially translatable within the elongate shaft in response to operation of the actuation mechanism. The elongate shaft includes a distal port to receive a suture. The elongate shaft includes a transverse slot extending inward from an outer surface of the elongate shaft generally perpendicular to the central longitudinal axis. The elongate shaft includes a suture lumen extending from the distal port axially within the elongate shaft to the transverse slot. The cutting blade intersects the transverse slot adjacent the suture lumen.

### CROSS-REFERENCE TO RELATED APPLICATIONS

This application claims the benefit of priority of U.S. Provisional Application No. 63/249,683 filed Sep. 29, 2021, the entire disclosure of which is hereby incorporated by reference.

### TECHNICAL FIELD

The present disclosure pertains to medical devices and methods for using medical devices. More particularly, the present disclosure pertains to aspects of medical devices for cutting a suture during a minimally invasive procedure such chordae repair.

### BACKGROUND

A wide variety of intracorporeal medical devices have been developed for medical use, for example, surgical and/or intravascular use. These medical devices are manufactured by any one of a variety of different manufacturing methods and may be used according to any one of a variety of methods. There is an ongoing need to provide alternative medical devices as well as alternative methods for manufacturing and/or using medical devices.

### SUMMARY

In one example, a medical device for cutting a suture during a minimally invasive procedure may comprise an elongate shaft having a proximal end, a distal end, and a central longitudinal axis extending from the proximal end the distal end; a handle disposed at the proximal end of the elongate shaft, the handle including an actuation mechanism; and a cutting blade disposed proximate the distal end of the elongate shaft. The cutting blade is axially translatable within the elongate shaft in response to operation of the actuation mechanism. The elongate shaft includes a distal port configured to receive a suture therein. The elongate shaft includes a transverse slot extending inward from an outer surface of the elongate shaft generally perpendicular to the central longitudinal axis. The elongate shaft includes a suture lumen extending from the distal port axially within the elongate shaft to the transverse slot. The cutting blade intersects the transverse slot adjacent the suture lumen.

In addition or alternatively to any example described herein, the elongate shaft includes a side port positioned generally opposite the transverse slot relative to the cutting blade.

In addition or alternatively to any example described herein, the transverse slot includes a first proximal wall facing toward the distal end of the elongate shaft. The side port includes a second proximal wall facing toward the distal end of the elongate shaft. The second proximal wall is disposed distal of the first proximal wall.

In addition or alternatively to any example described herein, the cutting blade includes a flattened main body portion oriented substantially parallel to the central longitudinal axis.

In addition or alternatively to any example described herein, the cutting blade includes a longitudinally oriented slot extending transversely therethrough.

In addition or alternatively to any example described herein, the cutting blade further includes a sharpened cutting edge proximate a distal end of the cutting blade and facing toward the proximal end of the elongate shaft.

In addition or alternatively to any example described herein, proximal axial translation of the cutting blade relative to the elongate shaft translates the sharpened cutting edge toward the transverse slot.

In addition or alternatively to any example described herein, the sharpened cutting edge is configured to cooperate with the transverse slot to cut a suture extending through the longitudinally oriented slot of the cutting blade.

In addition or alternatively to any example described herein, the elongate shaft includes a rounded distal cap secured to the distal end of the elongate shaft, wherein the rounded distal cap includes the distal port.

In addition or alternatively to any example described herein, and in another example, a medical device for cutting a suture during a minimally invasive procedure may comprise an elongate shaft having a proximal end, a distal end, and a central longitudinal axis extending from the proximal end the distal end; a handle disposed at the proximal end of the elongate shaft, the handle including an actuation mechanism; and a cutting blade disposed proximate the distal end of the elongate shaft. The actuation mechanism includes a pivoting linkage proximate the distal end of the elongate shaft, the pivoting linkage being coupled to the cutting blade. The cutting blade is axially translatable within the elongate shaft in response to operation of the actuation mechanism. The elongate shaft includes a distal port configured to receive a suture therein. The elongate shaft includes a suture lumen extending from the distal port proximally within the elongate shaft. The cutting blade is configured to intersect the suture lumen upon axial translation of the cutting blade from a first position to a second position.

In addition or alternatively to any example described herein, the cutting blade is slidably disposed within a longitudinally extending rectangular slot.

In addition or alternatively to any example described herein, the cutting blade includes a sharpened cutting edge facing distally.

In addition or alternatively to any example described herein, the medical device may include a polymeric block non-movably disposed within the longitudinally extending rectangular slot distal of the cutting blade.

In addition or alternatively to any example described herein, the pivoting linkage includes a central pivot point and a slot extending radially from the central pivot point and configured to slidably engage a pin coupled to the cutting blade.

In addition or alternatively to any example described herein, the suture is translatable within the suture lumen when the cutting blade is disposed in the first position.

In addition or alternatively to any example described herein, and in another example, a medical device for cutting a suture during a minimally invasive procedure may comprise an elongate shaft having a proximal end, a distal end, and a central longitudinal axis extending from the proximal end the distal end; a distal tip member fixedly attached to the distal end of the elongate shaft; and a cutting blade non-rotatably disposed within the distal tip member. The cutting blade is axially translatable within the distal tip member in response to operation of an actuation mechanism disposed proximate the proximal end of the elongate shaft. The distal tip member includes a distal port configured to receive a suture therein. The distal tip member includes a transverse slot extending radially inward from an outer surface of the distal tip member generally perpendicular to the central longitudinal axis, the transverse slot being at least partially defined by a first proximal wall facing distally. The distal tip member includes a suture lumen extending from the distal port axially within the distal tip member to the transverse slot. The distal tip member includes a side port positioned generally opposite the transverse slot, the side port being at least partially defined by a second proximal wall facing distally. The second proximal wall is axially offset from the first proximal wall along the central longitudinal axis.

In addition or alternatively to any example described herein, the second proximal wall is offset distally from the first proximal wall.

In addition or alternatively to any example described herein, the second proximal wall is oriented generally parallel to the first proximal wall.

In addition or alternatively to any example described herein, the cutting blade is disposed between the first proximal wall and the second proximal wall.

In addition or alternatively to any example described herein, when a suture extends within the suture lumen, into the transverse slot, through a longitudinally oriented slot in the cutting blade, and out the side port, and the cutting blade is translated proximally within the distal tip member, the suture biases the cutting blade away from the second proximal wall and toward the first proximal wall such that further proximal translation of the cutting blade within the distal tip member causes cooperation between a sharpened cutting edge of the cutting blade and the first proximal wall to cut the suture within the distal tip member.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cut-away view of an example heart and mitral valve;

FIGS. 2-6 illustrates an example process for implanting a chordae repair assembly;

FIG. 7 illustrates a configuration having more than one chordae repair assembly implanted;

FIG. 8 illustrates an example configuration of a medical device associated with the disclosure;

FIG. 9 illustrates an example configuration of a medical device associated with the disclosure;

FIGS. 10-12 illustrate selected aspects related to the construction and use of the medical device associated with the disclosure;

FIG. 13 illustrate aspects of an alternative configuration of a medical device associated with the disclosure;

FIGS. 14-15 illustrate selected aspects related to the construction and use of the medical device associated with the disclosure;

FIGS. 16-17 illustrate selected aspects related to the construction and use of an alternative configuration of the medical device associated with the disclosure;

FIG. 18 illustrates selected aspects related to a handle associated with the medical device;

FIG. 19 illustrates selected aspects of an alternative configuration of the handle associated with the medical device; and

FIG. 20 is a partial cross-sectional view of the handle of FIG. 19.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

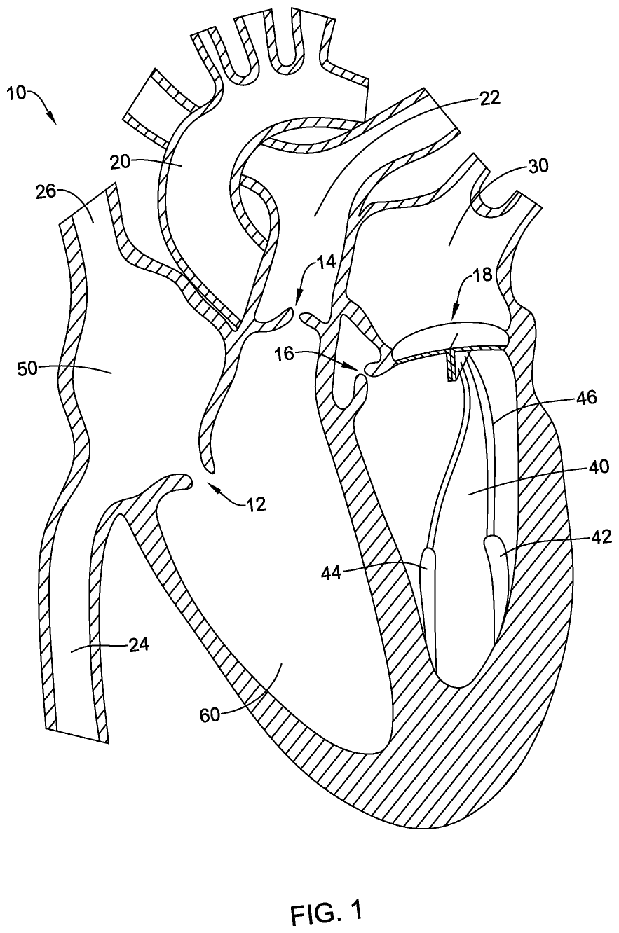

Diseases and/or medical conditions that impact the cardiovascular system are prevalent throughout the world. Some mammalian hearts (e.g., human, etc.) include four heart valves: a tricuspid valve 12, a pulmonary valve 14, an aortic valve 16, and a mitral valve 18, as seen in an example heart 10 illustrated in partial cross-section in FIG. 1. The purpose of the heart valves is to control blood flow into the heart 10 from major veins (e.g., the inferior vena cava 24, the superior vena cava 26, etc.), through the heart 10 (from atria to ventricles), and out of the heart 10 into the major arteries connected to the heart 10 (e.g., the aorta 20, the pulmonary artery 22, etc.). Each heart valve may have a plurality of valve leaflets configured to shift between an open configuration permitting fluid flow through the heart valve and a closed configuration wherein free edges of the valve leaflets coapt to substantially prevent fluid flow through the heart valve. The heart 10 may also include a left atrium 30, a left ventricle 40, a right atrium 50, and a right ventricle 60. The left ventricle 40 may include a first papillary muscle 42 attached to and/or extending from a wall of the left ventricle 40, a second papillary muscle 44 attached to and/or extending from the wall of the left ventricle 40, and a plurality of chordae tendineae 46 connecting the first papillary muscle 42 and the second papillary muscle 44 to the plurality of leaflets of the mitral valve 18. In a normally functioning heart valve, blood is permitted to pass or flow downstream through the heart valve (e.g., from an atrium to a ventricle, from a ventricle to an artery, etc.) when the heart valve is open (e.g., during diastole), and when the heart valve is closed (e.g., during systole), blood is prevented from passing or flowing back upstream through the heart valve (e.g., from a ventricle to an atrium, etc.).

In some instances, when mitral regurgitation occurs, a heart valve (e.g., the mitral valve 18) fails to open and/or close properly such that blood is permitted to pass or flow back upstream through the heart valve (e.g., from a ventricle to an atrium, etc.). In some cases, the defective heart valve may have leaflets that may not close, or may not be capable of closing, completely. In some instances, secondary or functional mitral regurgitation may be a secondary effect of left ventricular dysfunction, where left ventricular dilatation and/or distension caused by ischemic or idiopathic cardiomyopathy, for example, results in annular dilatation and/or distension of the left ventricle 40 and papillary muscle displacement with subsequent leaflet tethering and insufficient coaptation of the mitral leaflets during systole. In some instances, degenerative mitral regurgitation may involve redundant excessive tissue in part of the heart valve and/or the heart valve leaflets (e.g., mitral valve prolapse). In some instances, mitral regurgitation may be caused or exacerbated by stretching and/or rupture of one of more of the plurality of chordae tendineae 46.

Surgical methods of treating stretched or ruptured chordae tendineae may include replacing the chordae by sewing one or more sutures (e.g., Gore-Tex®, etc.) to the first papillary muscle 42 and/or the second papillary muscle 44 and to one or more leaflets of the plurality of valve leaflets to mimic the natural chordae tendineae. However, open heart cardiac surgery may carry significant risk to the patient, including complications, disability during recovery, and/or morbidity. Minimally invasive solutions may include transcatheter artificial valve replacement, but valve replacement surgeries may require lifelong anticoagulant treatment. Another alternative solution may involve edge-to-edge fixation of the plurality of leaflets, but such treatments prevent the option of minimally invasive valve replacement surgery in the future. As such, there is a need for minimally invasive treatments for repairing a heart valve while maintaining the option for future treatment options.

Disclosed herein are medical device(s) and/or method(s) that may be used to diagnose, treat, and/or repair a portion of the cardiovascular system. One possible remedy is a percutaneous procedure which may replace stretched and/or ruptured chordae tendineae. The disclosed medical device(s) and/or method(s) may preferably be used percutaneously via minimally invasive intravascular techniques, or in an alternative method, using open-heart surgical techniques. The medical device(s) and method(s) disclosed herein may also provide a number of additional desirable features and/or benefits as described in more detail below. For the purpose of this disclosure, the discussion below is directed toward repairing the plurality of chordae tendineae 46 attached to the mitral valve 18 and will be so described in the interest of brevity. This, however, is not intended to be limiting as the skilled person will recognize that the following discussion may also apply to another heart valve with no or minimal changes to the structure and/or scope of the disclosure. Additionally, the disclosure is described in the singular context of repairing one of the plurality of chordae tendineae 46 in the interest of brevity but may be applied to repairing more than one of the plurality of chordae tendineae 46 within the scope of the disclosure.

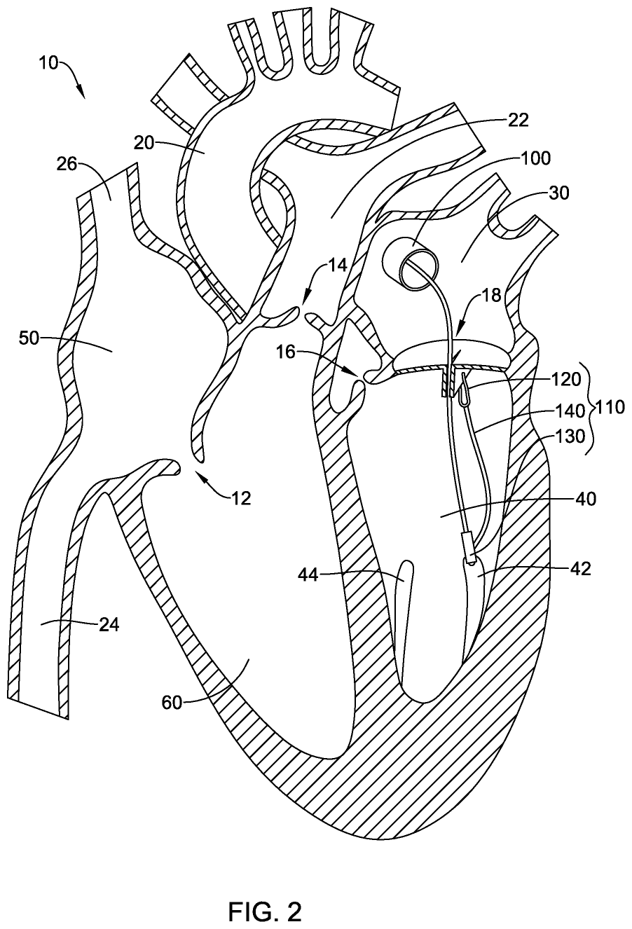

FIGS. 2-7 illustrates aspects of a percutaneous and/or minimally invasive method of implanting artificial chordae in the left ventricle 40. Some aspects of the method may be performed according to techniques known in the art, and thus are not described in great detail. As seen in FIG. 2, the method may include accessing the left atrium 30 and/or the mitral valve 18 using a delivery catheter 100. In some embodiments, the delivery catheter 100 may access the left atrium 30 and/or the mitral valve 18 transeptally. In some embodiments, the delivery catheter 100 may access the mitral valve 18 via a transaortic approach. Other approaches and/or configurations are also contemplated. Some suitable but non-limiting materials for the delivery catheter 100, for example metallic materials, polymer materials, composite materials, etc., are described below.

Next, a chordae repair assembly 110 may be implanted into the heart 10 from the delivery catheter 100. In at least some embodiments, the chordae repair assembly 110 may be implanted into the left ventricle 40 of the heart 10. In some embodiments, the chordae repair assembly 110 may include a leaflet grasping element 120, a ventricle anchor 130, and a suture 140 connecting the leaflet grasping element 120 to the ventricle anchor 130. In some embodiments, additional elements may also be included in the chordae repair assembly 110.

The leaflet grasping element 120 may be attached to one of the plurality of valve leaflets of the mitral valve 18 using a known delivery device and/or technique, such as a first catheter advanced through the delivery catheter 100. In some embodiments, the leaflet grasping element 120 may be attached to one of the plurality of valve leaflets of the mitral valve 18 from within the left ventricle 40. In some embodiments, the leaflet grasping element 120 may be attached to a free edge of one of the plurality of valve leaflets of the mitral valve 18. In at least some embodiments, the leaflet grasping element 120 may be fixedly attached to the free edge of one of the plurality of valve leaflets of the mitral valve 18. In some embodiments, the leaflet grasping element 120 may be removably attached to the free edge of one of the plurality of valve leaflets of the mitral valve 18.

In some embodiments, the leaflet grasping element 120 may include a spring clip configured to pinch the free edge of one of the plurality of valve leaflets of the mitral valve 18. In some embodiments, the leaflet grasping element 120 may include a ratcheting clip mechanism, a threaded anchoring element, or another type of element configured to be attached to the free edge of one of the plurality of valve leaflets of the mitral valve 18. Other configurations are also contemplated. Some suitable but non-limiting materials for the leaflet grasping element 120 and/or other associated components, for example metallic materials, polymer materials, composite materials, etc., are described below.

In some embodiments, medical imaging may be used to facilitate placement of the leaflet grasping element 120 and to verify placement location of the leaflet grasping element 120. In some embodiments, medical imaging may be used to verify quality of attachment of the leaflet grasping element 120 to one of the leaflets of the plurality of leaflets of the mitral valve 18. If the placement location and/or quality of attachment of the leaflet grasping element 120 is unsatisfactory, the leaflet grasping element 120 may be removed and either redeployed or replaced with a different leaflet grasping element.

The ventricle anchor 130 may be attached to tissue in the left ventricle 40 using a known delivery device and/or technique, such as a second catheter advanced through the delivery catheter 100 into the heart 10. In some embodiments, the ventricle anchor 130 may be attached to the first papillary muscle 42, as shown in FIG. 2. In some embodiments, the ventricle anchor 130 may be attached to the second papillary muscle 44. In some embodiments, the ventricle anchor 130 may be attached to a wall of the left ventricle 40. As discussed above, more than one chordae repair assembly 110 may be used in some procedures, and in such procedures, ventricle anchor(s) may be attached to at least one of, and possibly more than one of, the first papillary muscle 42, the second papillary muscle 44, and/or the wall of the left ventricle 40. In some embodiments, the ventricle anchor 130 may be fixedly attached to tissue in the left ventricle 40. In some embodiments, the ventricle anchor 130 may be fixedly attached to the first papillary muscle 42. In some embodiments, the ventricle anchor 130 may be fixedly attached to the second papillary muscle 44. In some embodiments, the ventricle anchor 130 may be fixedly attached to a wall of the left ventricle 40. In some embodiments, the ventricle anchor 130 may be removably attached to tissue in the left ventricle 40. In some embodiments, the ventricle anchor 130 may be removably attached to the first papillary muscle 42. In some embodiments, the ventricle anchor 130 may be removably attached to the second papillary muscle 44. In some embodiments, the ventricle anchor 130 may be removably attached to a wall of the left ventricle 40. Other configurations are also contemplated. Some suitable but non-limiting materials for the ventricle anchor 130 and/or other associated components, for example metallic materials, polymer materials, composite materials, etc., are described below.

In some embodiments, medical imaging may be used to facilitate placement of the ventricle anchor 130 and to verify placement location of the ventricle anchor 130. In some embodiments, medical imaging may be used to verify quality of attachment of the ventricle anchor 130 to the tissue in the left ventricle 40. If the placement location and/or quality of attachment of the ventricle anchor 130 is unsatisfactory, the ventricle anchor 130 may be removed and either redeployed or replaced with a different ventricle anchor.

As may be seen in FIG. 2, following placement of the leaflet grasping element 120 and the ventricle anchor 130, the suture 140 may extend from the leaflet grasping element 120, through the ventricle anchor 130, and into the delivery catheter 100. In some embodiments, the suture 140 may be a surgical suture as known in the art. In some embodiments, the suture 140 may be and/or may include a filament, a strand, a wire, a thread, or other flexible member. In some embodiments, the suture 140 may be substantially inelastic in an axial direction (e.g., longitudinally). As such, the suture 140 may be adapted, configured, and/or constructed to substantially avoid or prevent axial stretching. In some embodiments, the suture 140 may be configured to permit limited axial stretch (e.g., less than 10%, less than 5%, less than 2%, etc.). Some suitable but non-limiting materials for the suture 140, for example metallic materials, polymer materials, composite materials, etc., are described below.

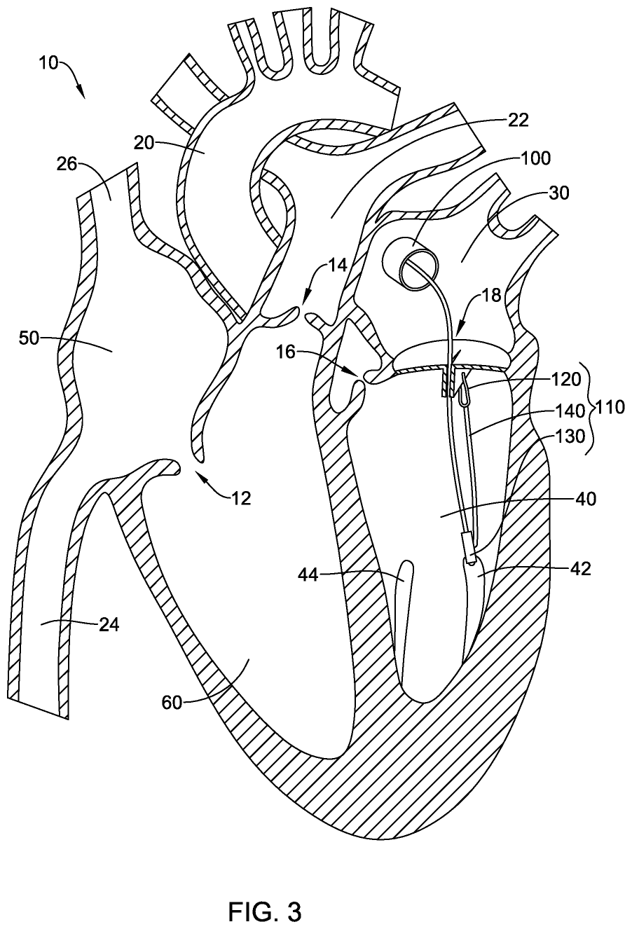

As shown in FIG. 3, tension on the suture 140 between the leaflet grasping element 120 and the ventricle anchor 130 may be adjusted as needed to provide adequate and/or proper operation of the plurality of leaflets of the mitral valve 18. For example, at least a portion of the suture 140 may be pulled proximally to remove slack therefrom. In some embodiments, at least a portion of the suture 140 may be pulled proximally to reduce a distance and/or adjust tension between the leaflet grasping element 120 and the ventricle anchor 130.

In some embodiments, the ventricle anchor 130 may be selectively lockable to and/or with respect to the suture 140. As such, the suture 140 may be configured to slide through the ventricle anchor 130 when the ventricle anchor 130 is in an unlocked configuration and the suture 140 may be secured relative to the ventricle anchor 130 when the ventricle anchor 130 is in a locked configuration.

After tension is applied to the suture 140, the ventricle anchor 130 may be shifted from the unlocked configuration to the locked configuration. With the ventricle anchor 130 in the locked configuration, the effect of the chordae repair assembly 110 on leaflet and valve function may be observed using medical imaging. Further tensioning and/or loosening of the suture 140 (via unlocking and re-locking the ventricle anchor 130, as needed) may be made until the desired function is achieved and/or obtained.

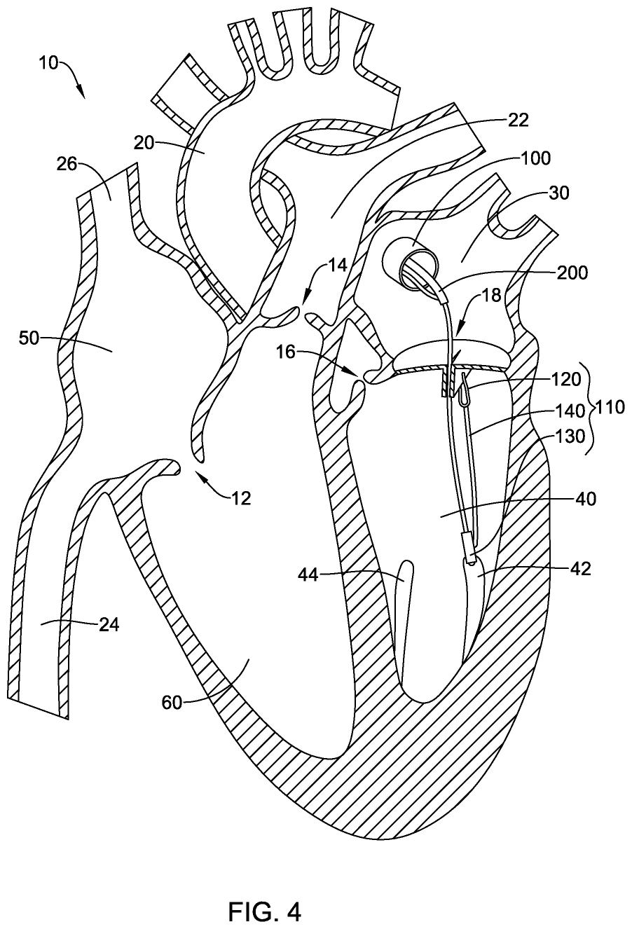

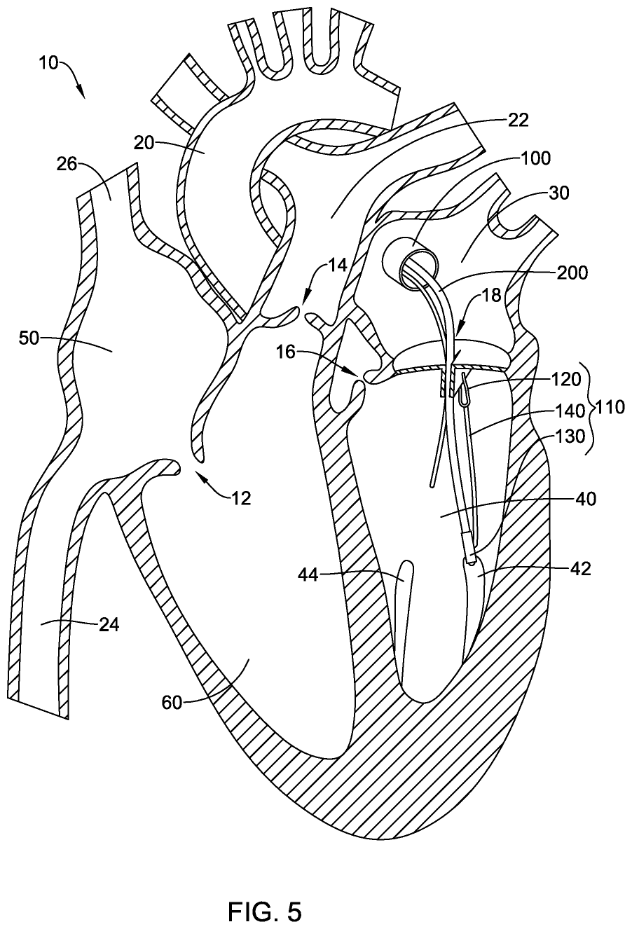

Next, a medical device 200 for cutting the suture 140 may be advanced over the suture 140 and/or through the delivery catheter 100, as seen schematically in FIG. 4. Additional details related to the medical device 200 will be discussed below. As shown in FIG. 5, the medical device 200 may be positioned adjacent a proximal end of the ventricle anchor 130 and the suture 140 may be cut using the medical device 200, as described herein.

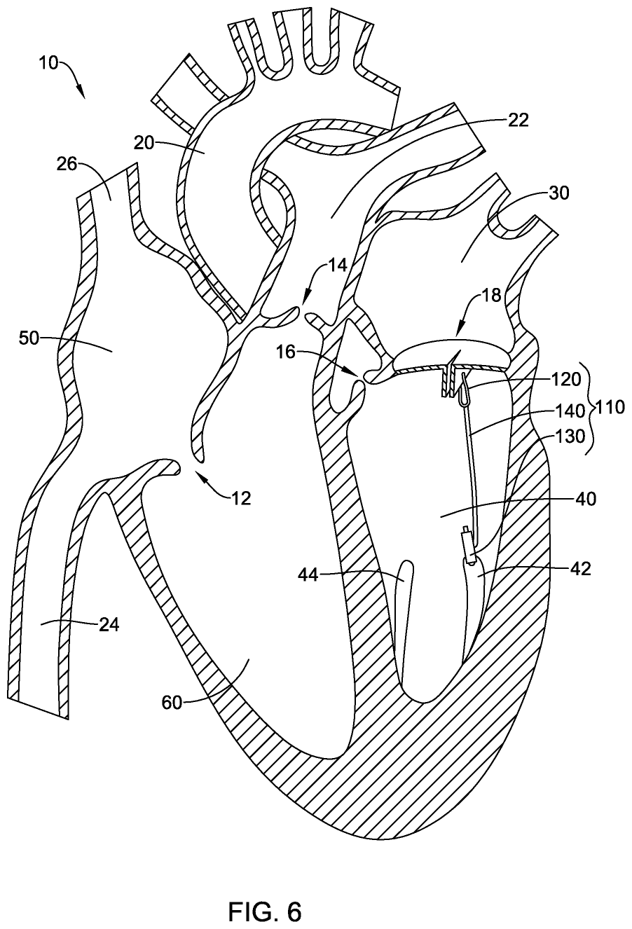

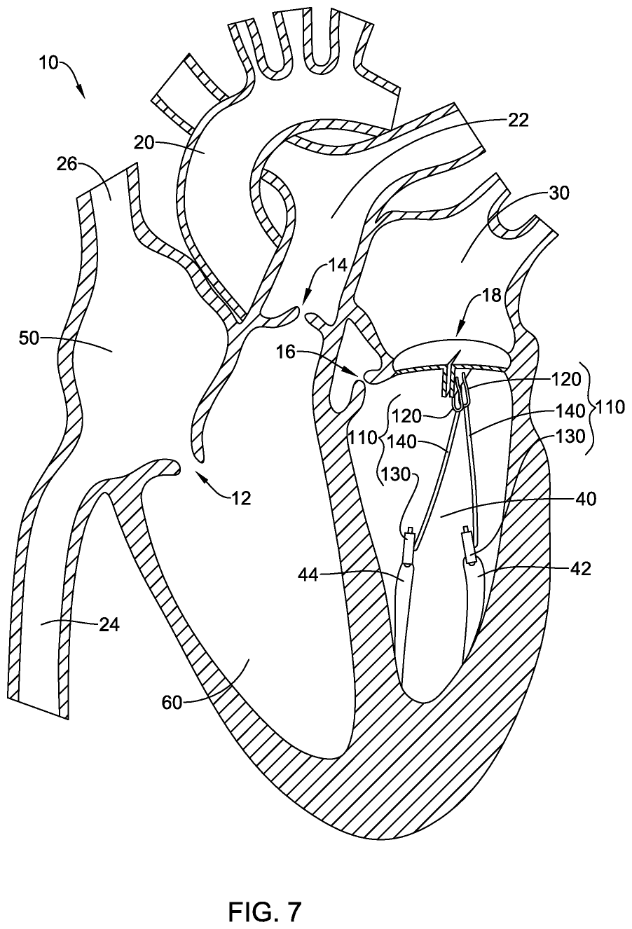

FIG. 6 illustrates the chordae repair assembly 110 in place within the heart 10. In some embodiments, the method and/or technique may require only one chordae repair assembly 110. In some embodiments, the method and/or technique may require a first chordae repair assembly 110 having a first ventricle anchor 130 attached to the first papillary muscle 42 and a second chordae repair assembly 110 having a second ventricle anchor 130 attached to the second papillary muscle 44, as shown in FIG. 7. In some embodiments, the first chordae repair assembly 110 and/or a first leaflet grasping element 120 may be spaced apart from the second chordae repair assembly 110 and/or the second leaflet grasping element 120. Other configurations are also contemplated. In at least some embodiments, placement of multiple chordae repair assemblies may be done through the same delivery catheter to minimize access points into the patient's vasculature and/or the heart.

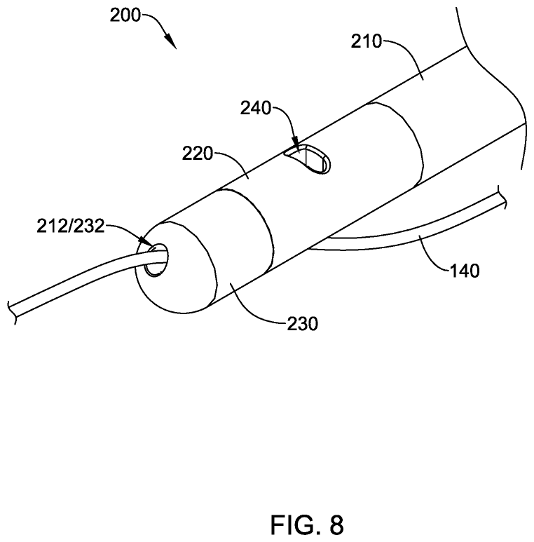

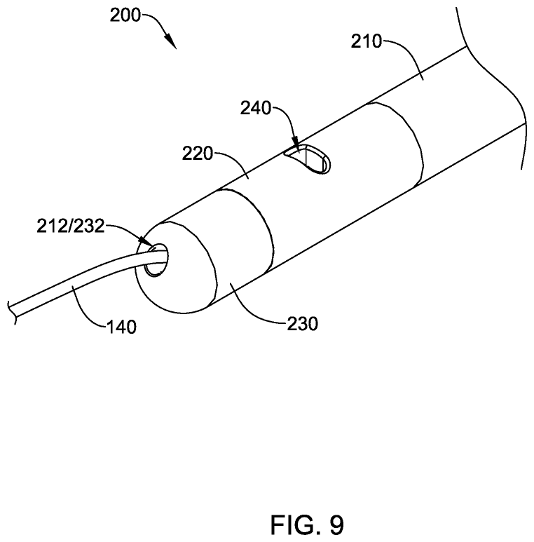

Returning briefly to FIGS. 5 and 6, the medical device 200 is shown in a “side shooter” or single operator exchange (SOE) configuration. In the “side shooter” or single operator exchange (SOE) configuration, the suture 140 may pass into a lumen of the medical device 200 at or near a distal end thereof and exit out a side of the medical device 200 and thereafter extend alongside the medical device 200 through the delivery catheter 100. FIG. 8 illustrates selected aspects of the medical device 200 in the “side shooter” or single operator exchange (SOE) configuration. In some configurations, the medical device 200 may have an internal or over-the-wire (OTW) configuration, some aspects of which are shown in FIG. 9. In the internal or over-the-wire (OTW) configuration, the suture 140 may pass into a lumen of the medical device 200 at or near the distal end thereof an extend internally an entire length of the medical device 200 to a proximal port or opening. Compared to the “side shooter” or single operator exchange (SOE) configuration, the internal or over-the-wire (OTW) configuration may require the suture 140 to have additional length to facilitate advancing the medical device 200 over the entire length of the suture 140. In the “side shooter” or single operator exchange (SOE) configuration, the suture 140 may be shorter because the medical device 200 only requires a short segment of the suture 140 to be passed through and/or inside of the medical device 200.

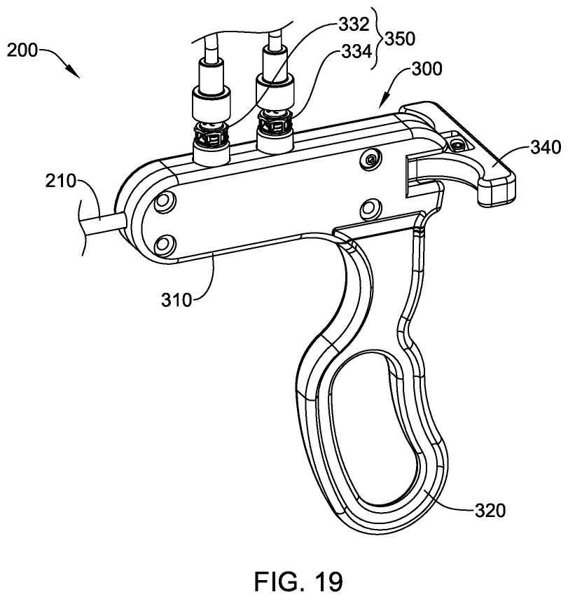

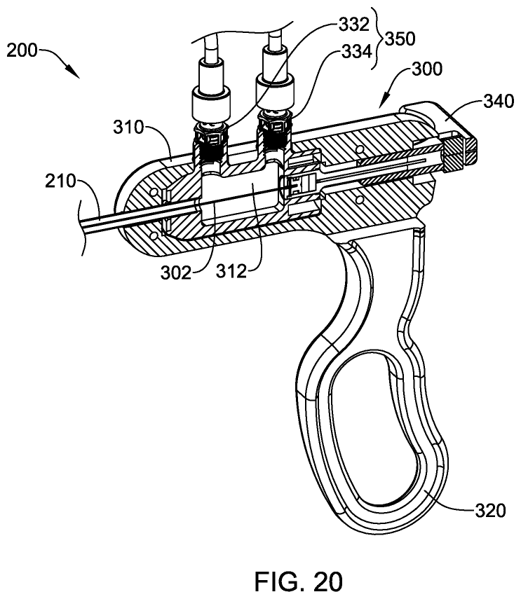

As seen in FIGS. 8-9, the medical device 200 may include an elongate shaft 210 having a proximal end, a distal end, and a central longitudinal axis extending from the proximal end to the distal end. In some embodiments, the medical device 200 may include a handle 300 (e.g., FIGS. 19-20) disposed at the proximal end of the elongate shaft 210. In some embodiments, the medical device 200 and/or the handle 300 may include an actuation mechanism, as described herein. In some embodiments, at least a portion of the actuation mechanism may be disposed proximate the proximal end of the elongate shaft 210.

In some embodiments, the medical device 200 and/or the elongate shaft 210 may include a distal tip member 220 fixedly attached to the distal end of the elongate shaft 210. In some embodiments, the distal tip member 220 may be integrally formed with the elongate shaft 210. In some embodiments, the distal tip member 220 may be constructed separately from the elongate shaft 210 and then fixedly attached to the elongate shaft 210. Some suitable but non-limiting materials for the elongate shaft 210 and/or the distal tip member 220, for example metallic materials, polymer materials, composite materials, etc., are described below.

In some embodiments, the elongate shaft 210 and/or the distal tip member 220 may include a distal port 212 configured to receive the suture 140 therein. In some embodiments, the elongate shaft 210 and/or the distal tip member 220 may include a rounded distal cap 230 secured to the distal end of the elongate shaft 210 and/or the distal tip member 220. In some embodiments, the rounded distal cap 230 includes a distal port 232. In some embodiments, the distal port 212 and the distal port 232 may be the same port. In some embodiments, the distal port 212 and the distal port 232 may be in fluid communication with each other. Other configurations are also contemplated. Some suitable but non-limiting materials for the rounded distal cap 230, for example metallic materials, polymer materials, ceramic materials, composite materials, etc., are described below.

The rounded distal cap 230 may be adapted, configured, and/or constructed to substantially avoid and/or prevent entanglement with the plurality of chordae tendineae 46 that may be intact and/or non-ruptured when working within the left ventricle 40 of the heart 10 (e.g., FIG. 1). In at least some embodiments, the distal port 212 and/or the distal port 232 may be laterally and/or radially offset from the central longitudinal axis of the elongate shaft 210 to facilitate axial translation of the cutting blade 260, as described herein.

In some embodiments, the elongate shaft 210 and/or the distal tip member 220 includes a transverse slot 240 extending radially inward from an outer surface of the elongate shaft 210 and/or an outer surface of the distal tip member 220 generally perpendicular to the central longitudinal axis of the elongate shaft 210. Additional details related to the transverse slot 240 are provided below.

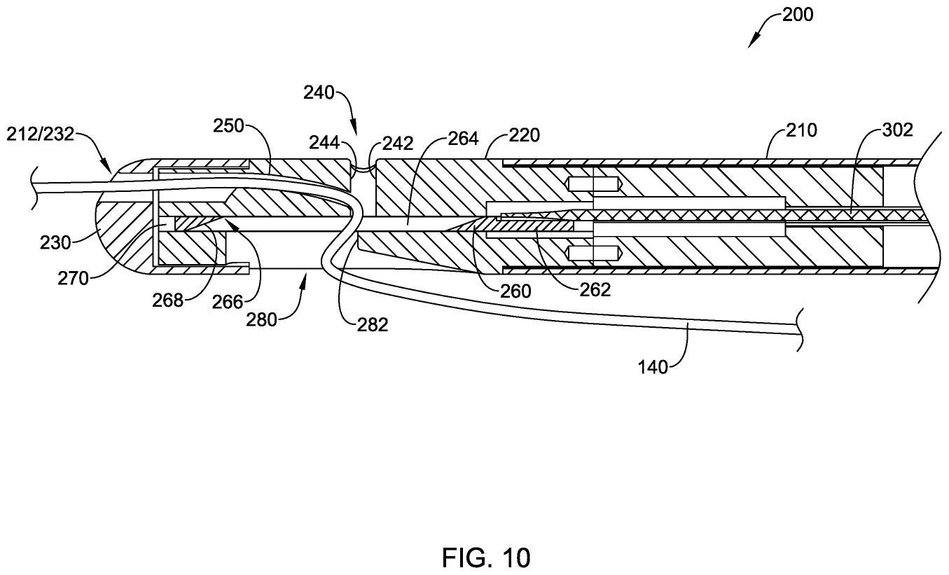

FIGS. 10-12 are partial cross-sectional views illustrating selected aspects related to the construction of the medical device 200 and selected aspects related to cutting the suture 140. In some embodiments, the elongate shaft 210 and/or the distal tip member 220 may include a suture lumen 250 extending proximally within the elongate shaft 210 and/or the distal tip member 220. In some embodiments, the suture lumen 250 may extend from the distal port 212 and/or the distal port 232 axially and/or proximally within the elongate shaft 210, the distal tip member 220, and/or the rounded distal cap 230. In some embodiments, the suture lumen 250 may extend from the distal port 212 and/or the distal port 232 axially and/or proximally within the elongate shaft 210, the distal tip member 220, and/or the rounded distal cap 230 to the transverse slot 240.

In some embodiments, the transverse slot 240 may include and/or may be at least partially defined by a first proximal wall 242 facing distally toward the distal end of the elongate shaft 210 and/or the distal tip member 220. The transverse slot 240 may include and/or may be at least partially defined by a first distal wall 244 facing proximally toward the proximal end of the elongate shaft 210 and/or the distal tip member 220. In some embodiments, the suture lumen 250 may open into the transverse slot 240 through the first distal wall 244.

In some embodiments, the medical device 200 may include a cutting blade 260 disposed proximate the distal end of the elongate shaft 210. In some embodiments, the cutting blade 260 may include a flattened main body portion 262 oriented substantially parallel to the central longitudinal axis of the elongate shaft 210. The flattened main body portion 262 of the cutting blade 260 may extend from a proximal end of the cutting blade 260 to a distal end of the cutting blade 260. In some embodiments, the cutting blade 260 may include a longitudinally oriented slot 264 extending transversely therethrough and/or extending transversely through the flattened main body portion 262 of the cutting blade 260. In some embodiments, the cutting blade 260 may include a sharpened cutting edge 266 proximate a distal end of the cutting blade 260. In some embodiments, the sharpened cutting edge 266 may face proximally toward the proximal end of the elongate shaft 210 and/or the distal tip member 220. In some embodiments, the sharpened cutting edge 266 may face distally toward the distal end of the elongate shaft 210 and/or the distal tip member 220. In some embodiments, the cutting blade 260 may include a ramp portion 268 extending from a full thickness of the flattened main body portion 262 toward the sharpened cutting edge 266. The ramp portion 268 may be oriented at an oblique angle to the flattened main body portion 262 and/or the central longitudinal axis of the elongate shaft 210.

In some embodiments, the cutting blade 260 may be slidably disposed within a longitudinally extending rectangular slot 270 formed within an interior of the elongate shaft 210 and/or the distal tip member 220. In some embodiments, the cutting blade 260 may be axially translatable within the elongate shaft 210 and/or the distal tip member 220 in response to operation of the actuation mechanism. In some embodiments, the cutting blade 260 may be axially translatable within the longitudinally extending rectangular slot 270 formed within the interior of the elongate shaft 210 and/or the distal tip member 220. In some embodiments, the cutting blade 260 may be axially translatable between a first position and a second position in response to operation of the actuation mechanism. In at least some embodiments, the cutting blade 260 may be non-rotatably disposed within the elongate shaft 210 and/or the distal tip member 220. Some suitable but non-limiting materials for the cutting blade 260, for example metallic materials, polymer materials, ceramic materials, composite materials, etc., are described below.

In some embodiments, the elongate shaft 210 and/or the distal tip member 220 may include a side port 280 positioned generally opposite the transverse slot 240 relative to the cutting blade 260. In some embodiments, the side port 280 includes and/or may be at least partially defined by a second proximal wall 282 facing distally toward the distal end of the elongate shaft 210 and/or the distal tip member 220. In some embodiments, the second proximal wall 282 is axially offset from the first proximal wall 242 along the central longitudinal axis of the elongate shaft 210. In some embodiments, the second proximal wall 282 is offset distally from the first proximal wall 242. In some embodiments, the second proximal wall 282 is disposed distal of the first proximal wall 242. In some embodiments, the second proximal wall 282 is oriented generally parallel to the first proximal wall 242.

In some embodiments, the second proximal wall 282 is spaced apart from the first proximal wall 242. In some embodiments, the cutting blade 260 is disposed between the first proximal wall 242 and the second proximal wall 282. In some embodiments, the second proximal wall 282 is spaced apart from the first proximal wall 242 by the cutting blade 260.

In some embodiments, the medical device 200 and/or the actuation mechanism may include a pull wire 302 extending proximally to the proximal end of the medical device 200 and/or the elongate shaft 210. In some embodiments, the pull wire 302 may be fixedly attached to the cutting blade 260. For example, the pull wire 302 may be welded, brazed, soldered, adhesively bonded, or otherwise permanently and fixedly attached to the cutting blade 260. In at least some embodiments, the pull wire 302 may be formed from a metallic material. Other materials and/or configurations are also contemplated. The pull wire 302 may be substantially inelastic and/or may be adapted, configured, and/or constructed to substantially avoid and/or prevent axial stretch. Some suitable but non-limiting materials for the pull wire 302, for example metallic materials, polymer materials, ceramic materials, composite materials, etc., are described below.

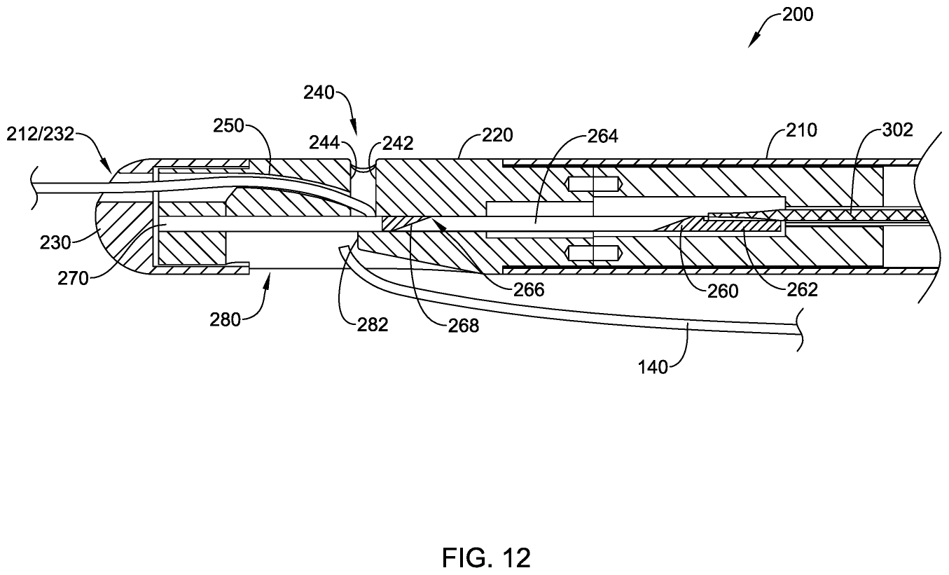

As may be seen in FIGS. 10-12, in some embodiments, the cutting blade 260 may intersect the transverse slot 240 adjacent the suture lumen 250. The suture 140 may be translatable within the suture lumen 250 and/or relative to the elongate shaft 210 and/or the distal tip member 220 when the cutting blade 260 is disposed in the first position. In some embodiments, the first position of the cutting blade 260 may be a distal position, seen in FIG. 10, and the second position of the cutting blade 260 may be a proximal position, seen in FIG. 12.

As shown in FIGS. 10-12, proximal axial translation of the cutting blade 260 relative to the elongate shaft 210 and/or the distal tip member 220 and/or within the longitudinally extending rectangular slot 270 formed within an interior of the elongate shaft 210 and/or the distal tip member 220 (via proximal translation of the pull wire 302, for example) may translate the sharpened cutting edge 266 of the cutting blade 260 toward the transverse slot 240 and/or the first proximal wall 242 that at least partially defines the transverse slot 240.

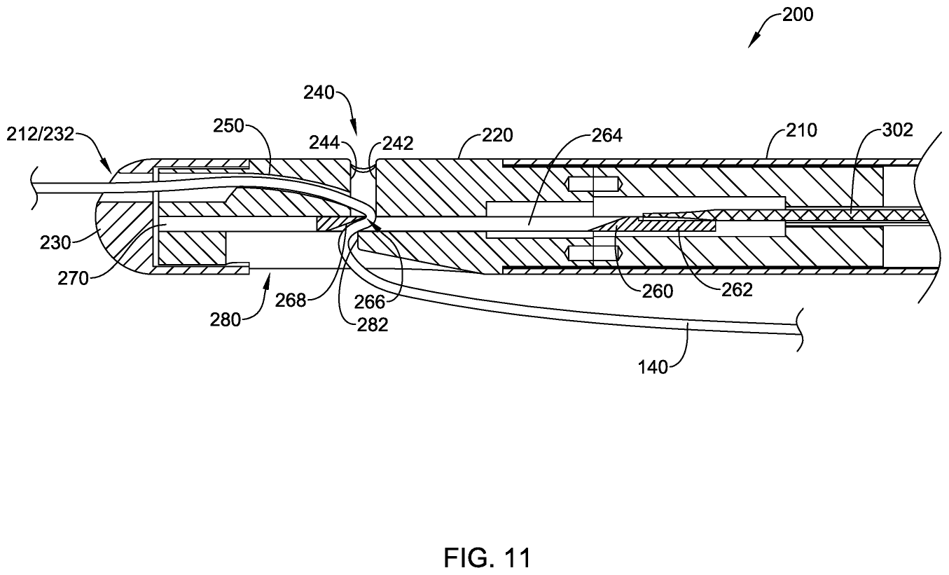

When the suture 140 extends within the suture lumen 250, into the transverse slot 240, through the longitudinally oriented slot 264 formed in the flattened main body portion 262 of the cutting blade 260, and out the side port 280, and the cutting blade 260 is in the first position, as shown in FIG. 10, the suture 140 may be axially translatable within the suture lumen 250, the longitudinally oriented slot 264, and the side port 280. When the suture 140 extends within the suture lumen 250, into the transverse slot 240, through the longitudinally oriented slot 264 formed in the flattened main body portion 262 of the cutting blade 260, and out the side port 280, and the cutting blade 260 is translated toward (e.g., proximally) the second position and/or relative to and/or within the elongate shaft 210 and/or the distal tip member 220, as shown in FIG. 11, the suture 140 may become pinched between the second proximal wall 282 and the ramp portion 268 of the cutting blade 260 such that the suture 140 may bias the sharpened cutting edge 266 of the cutting blade 260 away from the second proximal wall 282 and toward the first proximal wall 242 such that further axial and/or proximal translation of the cutting blade 260 relative to and/or within the elongate shaft 210 and/or the distal tip member 220 causes cooperation between the sharpened cutting edge 266 of the cutting blade 260 and the first proximal wall 242 to cut the suture 140 extending through the longitudinally oriented slot 264 of the cutting blade 260 within the elongate shaft 210 and/or the distal tip member 220, as shown in FIG. 12.

The design of the medical device 200 shown in FIGS. 10-12 may include, and/or the first proximal wall 242 and the second proximal wall 282 may define, a stepped offset through the longitudinally oriented slot 264 of the flattened main body portion 262 of the cutting blade 260. The ramp portion 268 and the second proximal wall 282 may cooperate to use the suture 140 itself bias the cutting blade 260 toward the first proximal wall 242, which forms and/or acts as a shearing surface. As a result of this configuration, biasing springs and/or super-tight tolerances are not necessary to achieve a clean cut of the suture 140. This may be particularly useful for suture materials that undergo at least some degree of compression before they can be or are cut. For example, some materials may have and/or include a number of air gaps within the material itself which may be compressed and/or squeezed before any cutting occurs. If too much gap or lateral movement between the cutting blade and the shearing surface (e.g., the first proximal wall) is present in the device, the suture may be squeezed and/or pinched between the surfaces without properly cutting, which could lead to stretching, thinning without cutting, tearing, material shaving or scraping, binding, excessive force requirements for axial translation of the cutting blade, etc.

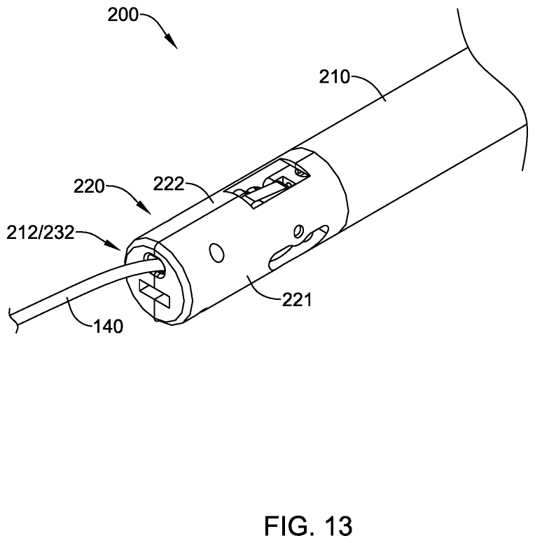

In some alternative embodiments, the medical device 200 may include a different configuration for the distal tip member 220 fixedly attached to the distal end of the elongate shaft 210, as seen in FIGS. 13-17 for example. While not expressly illustrated, it shall be understood that at least some embodiments of the medical device 200 shown in FIGS. 13-17 may include the rounded distal cap 230, as described herein. In some embodiments, the distal tip member 220 may include a first side portion 221 and a second side portion 222. Similar to above, the elongate shaft 210 and/or the distal tip member 220 may include a distal port 212. While the medical device 200 is expressly illustrated in FIGS. 13-17 as having an internal or over-the-wire (OTW) configuration, the medical device 200 may have a “side shooter” or single operator exchange (SOE) configuration, as described herein.

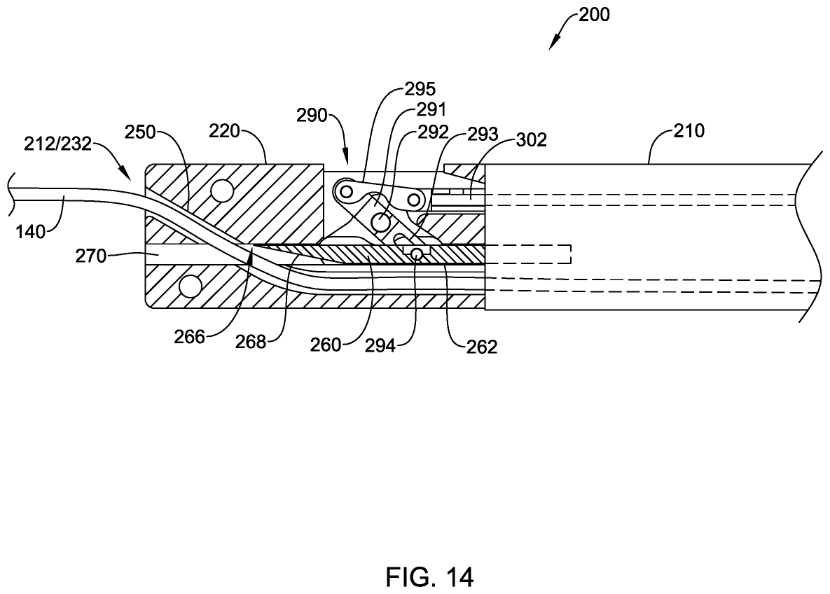

FIGS. 14-17 illustrate selected aspects related to the construction of the medical device 200 and selected aspects related to cutting the suture 140. In FIGS. 14-17, the first side portion 221 of the distal tip member 220 is not shown to improve understanding.

In some embodiments, the elongate shaft 210 and/or the distal tip member 220 may include a suture lumen 250 extending proximally within the elongate shaft 210 and/or the distal tip member 220. In some embodiments, the suture lumen 250 may extend from the distal port 212 and/or the distal port 232 (of the rounded distal cap 230, not shown) axially and/or proximally within the elongate shaft 210, the distal tip member 220, and/or the rounded distal cap 230.

In some embodiments, the medical device 200 may include a cutting blade 260 disposed proximate the distal end of the elongate shaft 210. In some embodiments, the cutting blade 260 may include a flattened main body portion 262 oriented substantially parallel to the central longitudinal axis of the elongate shaft 210. The flattened main body portion 262 of the cutting blade 260 may extend from a proximal end of the cutting blade 260 to a distal end of the cutting blade 260. In some embodiments, the cutting blade 260 may include a sharpened cutting edge 266 proximate a distal end of the cutting blade 260. In some embodiments, the sharpened cutting edge 266 may face proximally toward the proximal end of the elongate shaft 210 and/or the distal tip member 220. In some embodiments, the sharpened cutting edge 266 may face distally toward the distal end of the elongate shaft 210 and/or the distal tip member 220. In some embodiments, the cutting blade 260 may include a ramp portion 268 extending from a full thickness of the flattened main body portion 262 toward the sharpened cutting edge 266. The ramp portion 268 may be oriented at an oblique angle to the flattened main body portion 262 and/or the central longitudinal axis of the elongate shaft 210.

In some embodiments, the cutting blade 260 may be slidably disposed within a longitudinally extending rectangular slot 270 formed within an interior of the elongate shaft 210 and/or the distal tip member 220. In some embodiments, the cutting blade 260 may be axially translatable within the elongate shaft 210 and/or the distal tip member 220 in response to operation of the actuation mechanism. In some embodiments, the cutting blade 260 may be axially translatable within the longitudinally extending rectangular slot 270 formed within the interior of the elongate shaft 210 and/or the distal tip member 220. In some embodiments, the cutting blade 260 may be axially translatable between a first position and a second position in response to operation of the actuation mechanism. In at least some embodiments, the cutting blade 260 may be non-rotatably disposed within the elongate shaft 210 and/or the distal tip member 220. Some suitable but non-limiting materials for the cutting blade 260, for example metallic materials, polymer materials, ceramic materials, composite materials, etc., are described below.

In some embodiments, the actuation mechanism may include a pivoting linkage 290 proximate the distal end of the elongate shaft 210 and/or within the distal tip member 220. The pivoting linkage 290 may be movably coupled to the cutting blade 260. In some embodiments, the pivoting linkage 290 may be configured to move and/or actuate within the distal tip member 220 and/or within a distal end of the elongate shaft 210 to axially translate the cutting blade 260 within and/or relative to the elongate shaft 210 and/or the distal tip member 220.

In some embodiments, the pivoting linkage 290 may include at least one bar 291 configured to rotate about a central pivot point 292. In some embodiments, the central pivot point 292 may include and/or may be a pin, a shaft, a dowel, etc. In some embodiments, the pivoting linkage 290 and/or the at least one bar 291 may include a slot 293 extending radially from the central pivot point 292 and configured to slidably and/or pivotably engage a pin 294 coupled to the cutting blade 260 proximate and/or adjacent a first end of the at least one bar 291. In some embodiments, the pivoting linkage 290 may include a second bar member 295 pivotably coupled to a second end of the at least one bar 291 opposite the first end of the at least one bar 291.

In some embodiments, the medical device 200 and/or the actuation mechanism may include a pull wire 302 extending proximally to the proximal end of the medical device 200 and/or the elongate shaft 210. In some embodiments, the pull wire 302 may be coupled to the pivoting linkage 290. In some embodiments, the pull wire 302 may be movably and/or pivotably coupled to the pivoting linkage 290 and/or to the second bar member 295 of the pivoting linkage 290. In some embodiments, the pull wire 302 may be permanently and/or fixedly attached to at least a portion of the pivoting linkage 290. Other configurations are also contemplated. In at least some embodiments, the pull wire 302 may be formed from a metallic material. Other materials and/or configurations are also contemplated. The pull wire 302 may be substantially inelastic and/or may be adapted, configured, and/or constructed to substantially avoid and/or prevent axial stretch. Some suitable but non-limiting materials for the pull wire 302, the pivoting linkage 290, and/or elements thereof, for example metallic materials, polymer materials, ceramic materials, composite materials, etc., are described below.

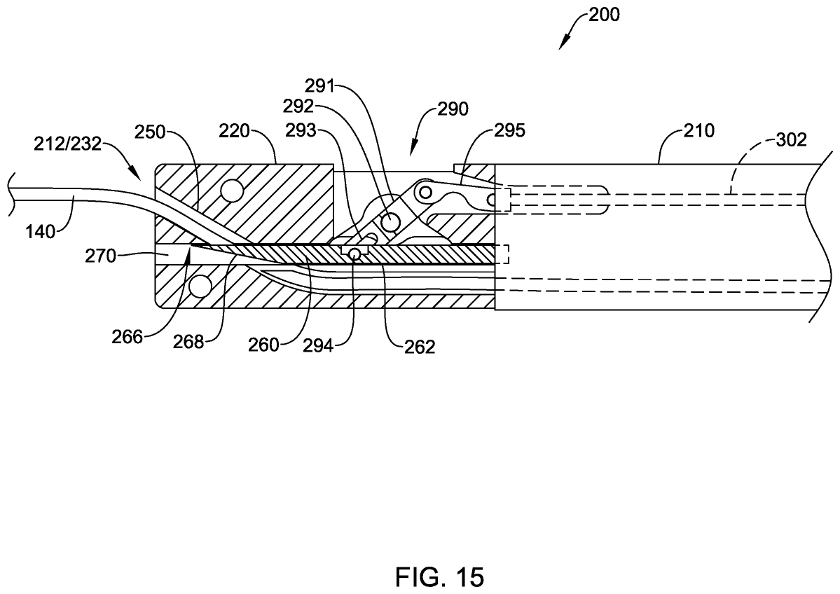

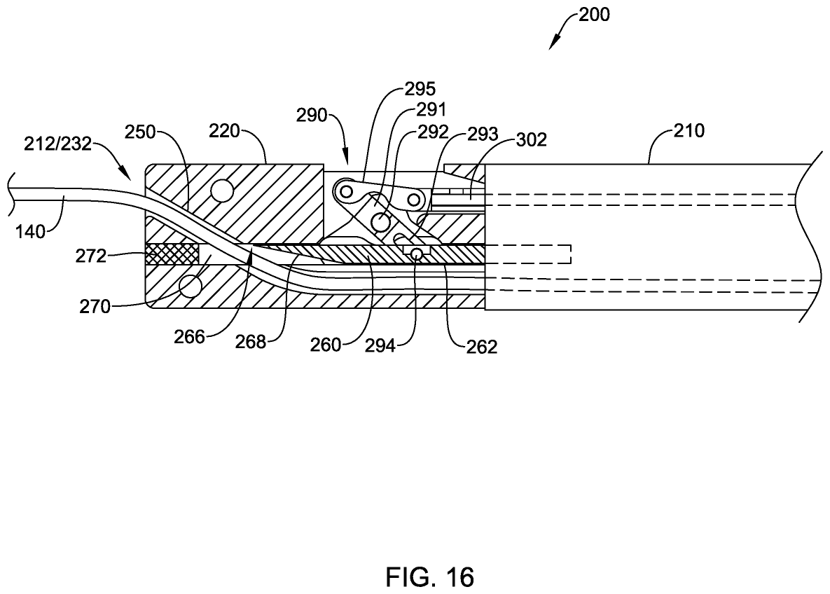

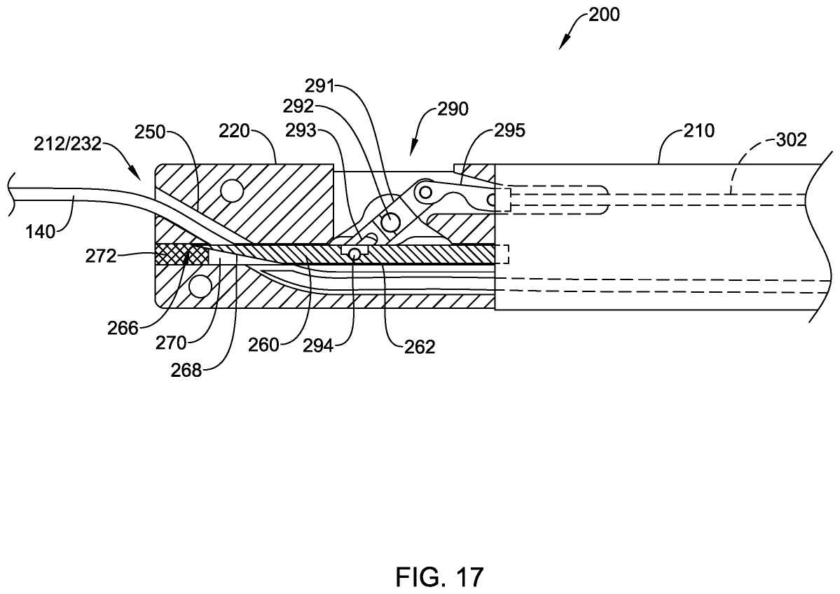

As may be seen in FIGS. 14-17, in some embodiments, the cutting blade 260 may be configured to intersect the suture lumen 250 upon axial translation of the cutting blade 260 from a first position to a second position. The suture 140 may be translatable within the suture lumen 250 and/or relative to the elongate shaft 210 and/or the distal tip member 220 when the cutting blade 260 is disposed in the first position. In some embodiments, the first position of the cutting blade 260 may be a proximal position, seen in FIGS. 14 and 16, and the second position of the cutting blade 260 may be a distal position, seen in FIGS. 15 and 17.

As shown in FIGS. 14-17, distal axial translation of the cutting blade 260 relative to the elongate shaft 210 and/or the distal tip member 220 and/or within the longitudinally extending rectangular slot 270 formed within an interior of the elongate shaft 210 and/or the distal tip member 220 (via proximal translation of the pull wire 302 and actuation of the pivoting linkage 290, for example) may translate the sharpened cutting edge 266 of the cutting blade 260 toward and/or through the suture lumen 250.

When the suture 140 extends within the suture lumen 250 and the cutting blade 260 is in the first position, as shown in FIGS. 14 and 16, the suture 140 may be axially translatable within the suture lumen 250. When the suture 140 extends within the suture lumen 250 and the cutting blade 260 is translated toward (e.g., distally) the second position and/or relative to and/or within the elongate shaft 210 and/or the distal tip member 220, as shown in FIGS. 15 and 17, the suture 140 may be cut by the cutting blade 260 and/or the sharpened cutting edge 266 of the cutting blade 260.

In some embodiments, the suture 140 may be cut by the cutting blade 260 and/or the sharpened cutting edge 266 of the cutting blade 260 when the cutting blade 260 and/or the sharpened cutting edge 266 of the cutting blade 260 passes completely through the suture lumen 250, as seen in FIGS. 14-15.

In some alternative embodiments, the medical device 200 may include a polymeric block 272 non-movably disposed within the longitudinally extending rectangular slot 270. In some embodiments, the polymeric block 272 may be fixedly secured within the longitudinally extending rectangular slot 270. In some embodiments, the polymeric block 272 may “catch” or receive the sharpened cutting edge 266 of the cutting blade 260 as the cutting blade 260 is axially translated distally and/or toward the second position. The polymeric block 272 may function as a shearing surface that cooperates with the sharpened cutting edge 266 of the cutting blade 260 to cut the suture 140 as and/or when the cutting blade 260 and/or the sharpened cutting edge 266 of the cutting blade 260 passes through the suture lumen 250, as seen in FIGS. 16-17. Some suitable but non-limiting materials for the polymeric block 272 are described below.

In some embodiments, the medical device 200 may include a handle 300. In some embodiments, the handle 300 may include an actuation mechanism or at least a portion of an actuation mechanism. In some embodiments, the handle 300 may include a handle body 310 secured and/or attached to the proximal end of the elongate shaft 210. In some embodiments, the handle body 310 may be fixedly attached to the proximal end of the elongate shaft 210. The elongate shaft 210 may extend distally from the handle body 310. In some embodiments, the handle 300 may include a grip 320 fixedly attached to the handle body 310. In some embodiments, the handle 300 may include one or more ports 330 attached to the handle body 310 and in fluid communication with the elongate shaft 210.

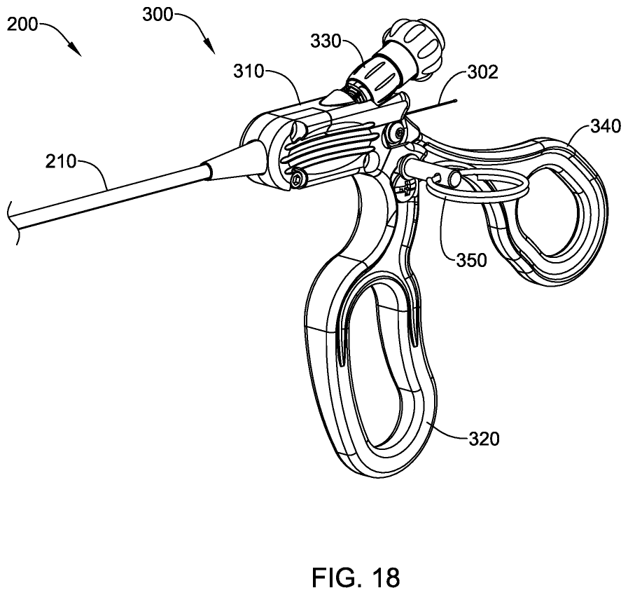

In some embodiments, shown in FIG. 18, the handle 300 may include an actuation lever 340 that is movable relative to the handle body 310. In some embodiments, the actuation lever 340 may be pivotably attached to the handle body 310. In some embodiments, the actuation lever 340 may be secured to the pull wire 302. In some embodiments, the actuation lever 340 may be fixedly attached to the pull wire 302. The actuation lever 340 may be configured to pivot relative to the handle body 310 toward the grip 320 from a first position to a second position to shift and/or axially translate the cutting blade 260 (e.g., FIGS. 10-12 and 14-17) from the first position to the second position by axially translating the pull wire 302 proximally within and/or relative to the elongate shaft 210. In some embodiments, the first position may be a proximal position and the second position may be a distal position. Other configurations are also contemplated. In some embodiments, the handle 300 may include a locking pin 350 or other locking element that is insertable into the handle body 310 when the actuation lever 340 is in the first position to prevent movement and/or actuation of the actuation lever 340 relative to the handle body 310 and/or the grip 320.

In some alternative embodiments, shown in FIGS. 19-20, the handle 300 may include an actuation lever 340 that is movable relative to the handle body 310. In some embodiments, the actuation lever 340 may be axially translatable relative to the handle body 310. In some embodiments, the actuation lever 340 may be secured to the pull wire 302. In some embodiments, the actuation lever 340 may be fixedly attached to the pull wire 302. The actuation lever 340 may be configured to axially translate and/or slide relative to the handle body 310 from a first position to a second position to shift and/or axially translate the cutting blade 260 (e.g., FIGS. 10-12 and 14-17) from the first position to the second position by axially translating the pull wire 302 proximally within and/or relative to the elongate shaft 210. In some embodiments, the first position may be a distal position engaged with and/or adjacent to the handle body 310, and the second position may be a proximal position spaced apart from the handle body 310. Other configurations are also contemplated. In some embodiments, the handle 300 may include a locking pin or other locking element that is insertable into the handle body 310 and/or attachable to the actuation lever 340 when the actuation lever 340 is in the first position to prevent movement and/or actuation of the actuation lever 340 relative to the handle body 310.

In some embodiments, the handle body 310 may include an internal chamber 312 in fluid communication with the one or more ports 330 attached to the handle body 310. In at least some embodiments, the pull wire 302 may pass through the internal chamber 312. In some embodiments, the internal chamber 312 may be in fluid communication with the elongate shaft 210. In some embodiments, the one or more ports 330 may include a first port 332 and a second port 334. In some embodiments, a fluid source may be connectable to the first port 332 and a vacuum source may be connectable to the second port 334, or vice versa. The fluid source may supply a fluid such as a saline solution or other biocompatible fluid into the internal chamber 312 and/or the elongate shaft 210 and the vacuum source may suction and/or remove air bubbles, debris, contamination, etc. from the internal chamber 312 and/or the elongate shaft 210. Other configurations are also contemplated. Some suitable but non-limiting materials for the handle 300, the handle body 310, the grip 320, and/or other associated components, for example metallic materials, polymer materials, composite materials, etc., are described below.

The materials that can be used for the various components of the medical device and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the delivery catheter, the elongate shaft, the pull wire, the handle, the distal tip member, the rounded distal cap, the cutting blade, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, a linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol, can be used to achieve desired properties.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system and/or components thereof in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system and/or components thereof to achieve the same result.

In some embodiments, a degree of Magnetic Resonance Imaging (Mill) compatibility is imparted into the system and/or other elements disclosed herein. For example, the system and/or components or portions thereof may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the Mill machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made to details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device for cutting a suture during a minimally invasive procedure, comprising:
an elongate shaft having a proximal end, a distal end, and a central longitudinal axis extending from the proximal end the distal end;
a handle disposed at the proximal end of the elongate shaft, the handle including an actuation mechanism; and
a cutting blade disposed proximate the distal end of the elongate shaft;
wherein the cutting blade is axially translatable within the elongate shaft in response to operation of the actuation mechanism;
wherein the elongate shaft includes a distal port configured to receive a suture therein;
wherein the elongate shaft includes a transverse slot extending inward from an outer surface of the elongate shaft generally perpendicular to the central longitudinal axis;
wherein the elongate shaft includes a suture lumen extending from the distal port axially within the elongate shaft to the transverse slot;
wherein the cutting blade intersects the transverse slot adjacent the suture lumen.

2. The medical device of claim 1, wherein the elongate shaft includes a side port positioned generally opposite the transverse slot relative to the cutting blade.

3. The medical device of claim 2, wherein the transverse slot includes a first proximal wall facing toward the distal end of the elongate shaft;
wherein the side port includes a second proximal wall facing toward the distal end of the elongate shaft;
wherein the second proximal wall is disposed distal of the first proximal wall.

4. The medical device of claim 1, wherein the cutting blade includes a flattened main body portion oriented substantially parallel to the central longitudinal axis.

5. The medical device of claim 4, wherein the cutting blade includes a longitudinally oriented slot extending transversely therethrough.

6. The medical device of claim 5, wherein the cutting blade further includes a sharpened cutting edge proximate a distal end of the cutting blade and facing toward the proximal end of the elongate shaft.

7. The medical device of claim 6, wherein proximal axial translation of the cutting blade relative to the elongate shaft translates the sharpened cutting edge toward the transverse slot.

8. The medical device of claim 6, wherein the sharpened cutting edge is configured to cooperate with the transverse slot to cut a suture extending through the longitudinally oriented slot of the cutting blade.

9. The medical device of claim 1, wherein the elongate shaft includes a rounded distal cap secured to the distal end of the elongate shaft, wherein the rounded distal cap includes the distal port.

10. A medical device for cutting a suture during a minimally invasive procedure, comprising:
an elongate shaft having a proximal end, a distal end, and a central longitudinal axis extending from the proximal end the distal end;
a handle disposed at the proximal end of the elongate shaft, the handle including an actuation mechanism; and
a cutting blade disposed proximate the distal end of the elongate shaft;
wherein the actuation mechanism includes a pivoting linkage proximate the distal end of the elongate shaft, the pivoting linkage being coupled to the cutting blade;
wherein the cutting blade is axially translatable within the elongate shaft in response to operation of the actuation mechanism;
wherein the elongate shaft includes a distal port configured to receive a suture therein;
wherein the elongate shaft includes a suture lumen extending from the distal port proximally within the elongate shaft;
wherein the cutting blade is configured to intersect the suture lumen upon axial translation of the cutting blade from a first position to a second position.

11. The medical device of claim 10, wherein the cutting blade is slidably disposed within a longitudinally extending rectangular slot.

12. The medical device of claim 11, wherein the cutting blade includes a sharpened cutting edge facing distally.

13. The medical device of claim 12, further including a polymeric block non-movably disposed within the longitudinally extending rectangular slot distal of the cutting blade.

14. The medical device of claim 10, wherein the pivoting linkage includes a central pivot point and a slot extending radially from the central pivot point and configured to slidably engage a pin coupled to the cutting blade.

15. The medical device of claim 10, wherein the suture is translatable within the suture lumen when the cutting blade is disposed in the first position.

16. A medical device for cutting a suture during a minimally invasive procedure, comprising:
an elongate shaft having a proximal end, a distal end, and a central longitudinal axis extending from the proximal end the distal end;
a distal tip member fixedly attached to the distal end of the elongate shaft; and
a cutting blade non-rotatably disposed within the distal tip member;
wherein the cutting blade is axially translatable within the distal tip member in response to operation of an actuation mechanism disposed proximate the proximal end of the elongate shaft;
wherein the distal tip member includes a distal port configured to receive a suture therein;
wherein the distal tip member includes a transverse slot extending radially inward from an outer surface of the distal tip member generally perpendicular to the central longitudinal axis, the transverse slot being at least partially defined by a first proximal wall facing distally;
wherein the distal tip member includes a suture lumen extending from the distal port axially within the distal tip member to the transverse slot;
wherein the distal tip member includes a side port positioned generally opposite the transverse slot, the side port being at least partially defined by a second proximal wall facing distally;
wherein the second proximal wall is axially offset from the first proximal wall along the central longitudinal axis.

17. The medical device of claim 16, wherein the second proximal wall is offset distally from the first proximal wall.

18. The medical device of claim 16, wherein the second proximal wall is oriented generally parallel to the first proximal wall.

19. The medical device of claim 16, wherein the cutting blade is disposed between the first proximal wall and the second proximal wall.

20. The medical device of claim 16, wherein when a suture extends within the suture lumen, into the transverse slot, through a longitudinally oriented slot in the cutting blade, and out the side port, and the cutting blade is translated proximally within the distal tip member, the suture biases the cutting blade away from the second proximal wall and toward the first proximal wall such that further proximal translation of the cutting blade within the distal tip member causes cooperation between a sharpened cutting edge of the cutting blade and the first proximal wall to cut the suture within the distal tip member.
